(19) United States
(12) Patent Application Publication
(10) Pub. No.: US 2009/0142347 A1
(43) Pub. Date: Jun. 4, 2009

(54) Tissue-Nonspecific Alkaline Phosphatase (TNAP): a Therapeutic Target for Arterial Calcification

(75) Inventor: José Luis Millan, La Jolla, CA (US)

Correspondence Address: CATALYST LAW GROUP, APC, 9710 SCRANTON ROAD, SUITE S-170, SAN DIEGO, CA, 92121, US

(73) Assignee: THE BURNHAM INSTITUTE FOR MEDICAL RESEARCH, La Jolla, CA (US)

(21) Appl. No.: 11/576,251

(22) PCT Filed: Sep. 29, 2005
(86) PCT No.: PCT/US05/35180

§ 371 Date: Mar. 28, 2007

Related U.S. Application Data
(60) Provisional application No. 60/614,758, filed on Sep. 29, 2004.

(51) Int. Cl. A61K 39/395; C07H 21/02; C12Q 1/42; C12Q 1/70; A61K 31/425; A61K 31/53; C07D 513/04; C07D 249/12; C07D 403/00

(52) U.S. Cl. 424/139.1; 536/23.2; 435/21; 435/5; 548/155; 548/264.4; 544/207; 514/368; 514/245

(57) ABSTRACT

This invention relates generally to the field of mineralization, and specifically to the role of TNAP in regulating the levels of extracellular inorganic pyrophosphate. The invention provides methods for modulating the activity of TNAP activity; methods for screening for modulators of TNAP activity; modulators of TNAP activity; and methods for treating pathologic conditions known of suspected to be affected by modulation of TNAP activity.

### PRIORITY APPLICATION INFORMATION

Benefit of priority under 35 U.S.C. 119(e) is claimed herein to U.S. Provisional Application No.: 60/614,758, filed Sep. 29, 2004. The disclosure of the above referenced application is incorporated by reference in its entirety herein.

### STATEMENT ON FEDERALLY SPONSORED RESEARCH

This invention was made in part with United States government support under grant numbers: RO1 DE12889 and RO1 AR47908, awarded by the National Institute of Health. The United States government has certain rights in this invention.

### FIELD OF THE INVENTION

This invention relates generally to the field of mineralization, and specifically to the role of TNAP in regulating the levels of extracellular inorganic pyrophosphate.

### BACKGROUND

The mechanisms that regulate tissue calcification are of major importance, as they ensure that calcification of the skeleton proceeds normally while mineralization is prevented elsewhere in the body. Alterations in these regulatory mechanisms, either due to genetic defects or as a result of aging, lead to disease, such as osteoarthritis and arterial calcification. Vascular calcification correlates clinically with the development of cardiovascular disease and atherosclerosis, and is also a common occurrence in aging, diabetes, renal failure, aortic stenosis and prosthetic valve replacement. Tissue calcification is an active process that is under the control of factors that regulate normal bone formation.

Inorganic pyrophosphate (PPi) is a potent inhibitor of calcification and three molecules have been identified as central regulators of mineralization via their ability to control the pool of extracellular PPi, (i.e., PPi that is generated, or transported to the, outside of the cells). Nucleotide pyrophosphatase phosphodiesterase 1 (NPP1) releases PPi from extracellular ATP. Ankylosis protein (ANK) transports PPi from the inside of the cell to the outside of the cell, while extracellular PPi is degraded to inorganic phosphate by the enzymatic action of tissue-nonspecific alkaline phosphatase (TNAP), an ectoplasmic enzyme.

Mice lacking NNP1 (Enpp1.sup.−/−) spontaneously develop articular cartilage, perispinal and aortic calcification. (Okawa et al., 1998; Johnson et al., 2003) These mice share similar phenotypic features with the human disease, Idiopathic Infantile Arterial Calcification (IIAC). In IIAC, a deficiency in NPP1-mediated production of PPi causes arterial calcification and periarticular calcifications of large joint. (Rutsch et al., 2001; Rutsch et al., 2003). Similarly, mice defective in the PPi channeling function of ANK (ank/ank), have decreased levels of extracellular PPi and display soft tissue ossification. (Ho et al., 2000).

Recent studies have determined that the major role for TNAP in bone tissue is hydrolysis of PPi to maintain proper levels of this mineralization inhibitor, thus insuring normal bone mineralization. (Johnson et al., 2000; Hessle et al., 2002; Johnson et al., 2003). Therefore, in a successful effort to normalize the levels of PPi in NPP1 null and ANK-deficient mice, and in turn correct soft tissue ossification abnormalities for these mice, Hessle et al., (2002) and Harmey et al., (2004) crossbred either the NPP1 null mice of the ANK-deficient mice with TNAP-deficient mice (Akp2^{-/-}). These genetic experiments reveal that the functional deletion of NPP1 and ANK lead to very similar disease states, including osteoarthritis, fusion of the ligaments of the spine, and arterial calcification, which can be normalized by the simultaneous interaction of TNAP gene.

Evidence points to the presence of alkaline phosphatase—(AP) rich vesicles at sites of mineralization in human arteries. It has been demonstrated that increased levels of TNAP (an alkaline phosphatase family member) accelerate calcification in bovine smooth muscle cells (VSMCs) and moreover, levamisole, a TNAP inhibitor blocks bovine VSMC calcification in a dose-dependent manner. (Shioi et al., 1995). The presence of TNAP-enriched matrix vesicles (MV) in human atherosclerotic lesions also suggests its active role in the promotion of vascular calcification. (Hsu and Camacho, 1999; Hui et al., 1997a; Hui and Tenenbaum, 1998; Tanimura et al., 1986a, b). MVs derived from primary osteoblasts from hypophosphatasia mice (Akp2^{-/-}) have increased levels of PPi. Recently, Mathieu et al. (2005) showed that calcification of human valve interstitial cells is dependent on AP activity.

Thus, there is a need in the art to further elucidate the molecular mechanisms involved in tissue mineralization. There is a further need in the art to modulate this mineralization process, thereby treating the resultant disorders. Therefore there is a need in the art for novel modulators of TNAP activity. Further still, there is a need in the art to diagnose these disorders based on dysregulation of and/or genetic abnormalities with the molecular components of the mineralization process.

### SUMMARY OF THE INVENTION

The invention provides methods for modulating (increasing or decreasing) TNAP activity. Modulation of TNAP activity affects a change in the concentration of extracellular PPi. Such methods include providing agents capable of modulating TNAP activity. Methods for modulation of TNAP activity can be used for modulation of TNAP in vivo and ex vivo. Moreover, the method of modulation can include pharmaceutical formulations of the modulators. Thus, the methods of modulation can include, but are not limited to methods of treating a disorder by modulating the activity of TNAP.

In a further aspect of this invention there is provided a method for screening for modulators of TNAP activity. In a particular embodiment of this screening method there is provided the specific TNAP modulation domain of the TNAP polypeptide. This domain can be utilized in a variety of systems, including, but not limited to native isolated TNAP polypeptides, fusion TNAP polypeptides, recombinant TNAP polypeptides, polypeptide fragments comprising at least the TNAP modulation domain, chimeric polypeptides comprising at least the TNAP modulation domain and combinations thereof.

Agents that are screened by this modulator screening method to determine their ability to modulate TNAP activity can include, but are not limited to, a peptide, polypeptide, peptidomimetic, non-peptidyl compound, carbohydrate, lipid, a synthetic compound, a natural product, an antibody or antibody fragment, a small organic molecule, a small inorganic molecule, and a nucleotide sequence. In one embodiment the screening method can be performed in vitro. Furthermore, the screening method can be performed as a High Throughput Screening assay (HTS). In an alternate embodiment, the screening method can be performed as a computational modeling study. In a still further embodiment, the screening method can be performed in vivo; for example employing animal models. Moreover, the screening method can be performed using transgenic cell lines. These various formats for performing the screening method of the current invention are not mutually exclusive, and as such can be used in combinations with one another.

In a further aspect of this current invention there are provided compositions useful for modulating TNAP activity. These compositions can include, but are not limited to, a peptide, polypeptide, peptidomimetic, non-peptidyl compound, carbohydrate, lipid, a synthetic compound, a natural product, an antibody or antibody fragment, a small organic molecule, a small inorganic molecule, a nucleotide sequence, and pharmaceutical formulations thereof.

In a further aspect of this invention there is provided a method for treating pathologic conditions using modulators of TNAP activity. In one embodiment of this treatment method there is provided an agent known to modulate TNAP activity. The agent can be a peptide, polypeptide, peptidomimetic, non-peptidyl compound, carbohydrate, lipid, a synthetic compound, a natural product, an antibody or antibody fragment, a small organic molecule, a small inorganic molecule, a nucleotide sequence, and pharmaceutical formulations thereof. In this aspect of the invention, the pathologic condition is treated using a modulator of TNAP activity is one known or suspected to be treated by the modulation of TNAP activity. In a particular illustration of this aspect of the invention, the pathologic condition is known or suspected to be affected by modulating the extracellular concentration of inorganic pyrophosphate.

In another embodiment of this treatment method TNAP activity is modulated utilizing gene therapy techniques. The gene therapy techniques can deliver to cells, tissue, organ or animal exogenous polynucleotides known to modulate TNAP activity. Exogenous polynucleotide sequences can be administered to a patient using an administration system comprising a nucleic acid vector system, microinjection, a gene gun and a liposome.

Such treatment methods are useful for treating human and non-human animals for, without limitation, aging, arthritis, aneurysm, atherosclerosis, diabetes, renal failure, aortic stenosis, prosthetic valve calcification, arterial calcification and cardiovascular disease.

In a further aspect of the current invention there is provided the TNAP modulation polypeptide domain and the polynucleotide domain as well as polypeptide and polynucleotide sequences substantially similar thereto. The TNAP modulation domain correlates with the specificity and selectivity of modulation by various modulators.

In one embodiment, this domain is used for in silico computational modeling for determining candidate modulator structures. In a further embodiment, this domain is used for developing in vivo modulator screening assays. In a still further embodiment, this domain is useful for developing gene therapy constructs. In a further embodiment, this domain is useful for developing agents for treating disorders known or suspected to respond to TNAP activity.

The TNAP modulation domain of the current invention comprises, isolated native polypeptide and polynucleotide sequences, full-length polypeptide and polynucleotide sequences, recombinant polypeptide and polynucleotide sequences, chimeric polypeptide and polynucleotide sequences, substituted polypeptide and polynucleotide sequences, and fragment polypeptide and polynucleotide sequences.

### BRIEF DESCRIPTION OF THE DRAWINGS

FIG. 1 illustrates normal uncalcified osteoid (Ost) layer (A) versus widened osteoid layer in TNAP-deficient tibial metaphyseal bone (B). A few intact MVs, containing apatite-like needles (indicated by arrows and shown at higher magnification in inserts) are present in the uncalcified osteoid of both TNAP wild-type and TNAP-deficient tibias. (M=mineralized bone matrix, Obl=osteoblast, Ost=osteoid) (A and B×25,000, A insert×61,000; B insert×127,000).

FIG. 2 depicts whole mount skeletal preparations of wild-type (WT), Enpp1−/− mice display a more severe soft tissue ossification phenotype than ank/ank mice. Larger arrows indicated the increased amount of mineral in the phalanges of the ank/ank mice (C). Small arrows indicate the areas of soft tissue mineralization in the metatarsal bones of Enpp1−/− mice (B, E). Ossification of the Achilles tendon is also observed (H, K).

FIG. 3 shows the correlation between serum PPi and OPN levels. The elevated levels of PPi in Akp2−/− mice cause a secondary increase in OPN, whereas the decreased PPi concentrations in Enpp1−/− and ank/ank mice result in depressed OPN levels. Therefore there is a strong correlation between PPi concentration and serum OPN levels. The double mutant mice, i.e., Akp2−/−; Enpp1−/− and Akp2−/−; ank/ank, show normalized PPi levels that also result in correction of OPN levels.

FIG. 4 presents the scheme depicting the roles of TNAP, ANK, NPP1, PPi and OPN in the regulation of hydroxyapaptite deposition. Both NPP1 and ANK raise extracellular levels of PPi while TNAP is required for depletion of the PPi pool. Both TNAP and NPP1 are functional in MVs whereas ANK is not. Therefore NPP1 plays more crucial role in PPi production than ANK. As a result, the absence of NPP1 in Enpp1−/− mice results in a more severe phenotype than ank/ank mice. A negative feedback loop exists in which PPi, produced by NPP1 and transported by the channeling action of ANK, inhibits expression of the Enpp1 and Ank genes. In addition PPi induces expression of the OPN gene and production of OPN, which further inhibits mineralization. In the absence of TNAP, high levels of PPi inhibit mineral deposition directly and also via its induction of OPN expression. The combined action of increased concentrations of PPi and OPN causes hypomineralization. In the absence of NPP1 or ANK, low levels of PPi, in addition to decrease in OPN levels, leads to ectopic calcification.

FIG. 5 shows in vitro culture of VSMCs. Left panel: A) immunofluorscent detection of smooth muscle alpha-actin. VSMCs were isolated from WT aorta by collagenase digestion, all cells were stained for actin filaments (F-actin) by rhodamine phalloidin (red), and VSMCs were stained using a specific FITC-conjugated monoclonal anti-SM-alpha-actin (green). B) Proportion of cells staining positive for SM-alpha-actin. C) Total RNA was isolated and SM alpha-actin was detected by RT-PCR analysis. Middle panel: VSMCs isolated from WT, Enpp1−/− and ank/ank mice were cultured in the presence of beta-glycerophosphate for 4 weeks. Cells were then stained for TNAP activity (pink) and using von Kossa staining, mineral was detected (black/brown). The area of the culture well in which mineralization was present was quantified by using a point-counting method in which the plate was placed on a grid (divided into 10 mm×10 mm squares) and visualized using a dissecting microscope, the percent area occupied by mineral was assessed by counting the occurrences where the presence of mineral coincided with intercepts on the grid. Right panel: Tetramisole treatment of VSMCs (+βGP) from wild-type, Enpp1−/−, and ank/ank mice cultured in the presence or absence of tetramisole (0.125 mM) for 3 weeks. The cells were then stained for TNAP activity, and then detached from the culture wells and the asorbance of the samples was measured at 405 nm and normalized to protein content.

FIG. 6 summarizes the quantification of the amount of calcium present in aortas or in serum of 3 month-old wild-type, Enpp1−/− and ank/ank mice.

FIG. 7 shows 3 month old wild-type and Enpp1−/− mice dissection from adherent tissue with the exception of the heart and aorta. The heart and aorta are digitally outlined on the image. The ribcage, heart, and aorta were fixed in 100% ethanol and unmineralized osteoid stained with Alcian blue followed by Alizarin red staining for mineralized osteoid. The samples were cleared in 2% KOH and stored in 100% glycerol. Panels A and B show low magnification images of the ribcage. The arrow points to several foci of calcification as revealed by positive staining (red) for calcium in the aorta in the Enpp1−/−sample. The foci are better observed at higher magnification in Panel D. In panels E and F the aorta has been dissected away from the spine and the presence of calcium deposits is clearly seen in the Enpp1−/− specimen.

FIG. 8 shows the calculated optimal docking of levamisole and theophylline into the modeled active site of TNAP (TNAP modulation domain). For the ligands, geometrical and non-bonded parameters were derived from ab inito quantum calculations with the program GAUSSIAN98.

FIG. 9 illustrates some novel lead compounds that inhibit TNAP activity.

FIG. 10 is a bar graph illustrating that Enpp1−/− and ank/ank mice show improvement in spine flexibility when treated with tetramisole. Flexibility was measured after euthanasia by determining the degree to which the root of the tail could be pulled back towards the neck, shoulders or lumbar spine until resistance prevented further flexing. The bar graph of FIG. 10 shows the results obtained for the lumbar spine (n=10 mice per group). Comparable results were obtained for neck and shoulder flexibility.

### DETAILED DESCRIPTION OF THE INVENTION

As used herein the singular forms “a”, “and”, and “the” include plural referents unless the context clearly dictates otherwise. For example, “a compound” refers to one or more of such compounds, while “the enzyme” includes a particular enzyme as well as other family members and equivalents thereof as known to those skilled in the art.

Generally, the nomenclature used hereafter and the laboratory procedures in cell culture, molecular genetics, and nucleic acid chemistry and hybridization described below are those well known and commonly employed in the art. Standard techniques are used for recombinant nucleic acid methods, polynucleotide synthesis, cell culture, and transgene incorporation (e.g., electroporation, microinjection, lipofection). Techniques and procedures are generally performed according to conventional methods in the art and various general references which are provided throughout this document, as well as: Maniatis et al., Molecular Cloning: A Laboratory Manual (1989), 2nd Ed., Cold Spring Harbor, N.Y.; and Berger and Kimmel, Methods in Enzymology, Volume 152, Guide to Molecular Cloning Techniques (1987), Academic Press, Inc., San Diego, Calif., which are incorporated herein by reference.

The term “polynucleotide” refers to a deoxyribonucleotide or ribonucleotide polymer in either single-or double-stranded form, and unless otherwise limited, would encompass known analogs of natural nucleotides that can function in a similar manner as naturally occurring nucleotides. A “polynucleotide sequence” also refers to a polynucleotide molecule or oligonucleotide molecule in the form of a separate fragment or as a component of a larger nucleic acid. The polynucleotide sequence may also be referred to as a “nucleotide probe.” Some of the polynucleotides of the invention are derived from DNA or RNA isolated at least once in substantially pure form and in a quantity or concentration enabling identification, manipulation, and recovery of its component nucleotide sequence by standard biochemical methods. Examples of such methods, including methods for PCR protocols that may be used herein, are disclosed in Sambrook et al. Molecular Cloning: A Laboratory Manual, 2nd ed., Cold Spring Harbor Laboratory Press, New York (1989), Ausubel, F. A., et al., eds., Current Protocols in Molecular Biology, John Wiley and Sons, Inc., New York (1987), and Innis, M., et al. (Eds.) PCR Protocols: A Guide to Methods and Applications, Academic Press, San Diego, Calif. (1990). Reference to a nucleic acid molecule also includes its complement as determined by the standard Watson-Crick base-pairing rules, with uracil (U) in RNA replacing thymine (T) in DNA, unless the complement is specifically excluded.

As described herein, the polynucleotides of the invention include DNA in both single-stranded and double-stranded form, as well as the DNA or RNA complement thereof. DNA includes, for example, DNA, genomic DNA, chemically synthesized DNA, DNA amplified by PCR, and combinations thereof. Genomic DNA, including translated, non-translated and control regions, may be isolated by conventional techniques, e.g., using any one of the cDNAs of the invention, or suitable fragments thereof, as a probe, to identify a piece of genomic DNA which can then be cloned using methods commonly known in the art.

Polypeptides encoded by the polynucleotides of the invention are encompassed by the invention. As used herein, reference to a nucleic acid “encoding” a protein or polypeptide encompasses not only cDNAs and other intronless nucleic acids, but also DNAs, such as genomic DNA, with introns, on the assumption that the introns included have appropriate splice donor and acceptor sites that will ensure that the introns are spliced out of the corresponding transcript when the transcript is processed in a eukaryotic cell. Due to the degeneracy of the genetic code wherein more than one codon can encode the same amino acid, multiple DNA sequences can code for the same polypeptide. Such variant DNA sequences can result from genetic drift or artificial manipulation (e.g., occurring during PCR amplification or as the product of deliberate mutagenesis of a native sequence). Deliberate mutagenesis of a native sequence can be carried out using numerous techniques well known in the art. For example, oligonucleotide-directed site-specific mutagenesis procedures can be employed, particularly where it is desired to mutate a gene such that predetermined restriction nucleotides or codons are altered by substitution, deletion or insertion. Exemplary methods of making such alterations are disclosed by Walder et al. (Gene 42:133,1986); Bauer et al. (Gene 37:73, 1985); Craik (BioTechniques, Jan. 12-19, 1985); Smith et al. (Genetic Engineering: Principles and Methods, Plenum Press, 1981); Kunkel (Proc. Natl. Acad. Sci. USA 82:488, 1985); Kunkel et al. (Methods in Enzymol. 154.367, 1987). The present invention thus encompasses any nucleic acid capable of encoding a protein of the current invention.

The phrase “substantially identical” means that a relevant polynucleotide or polypeptide sequence is at least 70%, 75%, 80%, 85%, 90%, 92%, 95% 96%, 97%, 98%, or 99% identical to a given SEQ ID NO. By way of example, such sequences may be allelic variants, sequences derived from various species, or they may be derived from the given sequence by truncation, deletion, amino acid substitution or addition. Percent identity between two sequences is determined by standard alignment algorithms such as ClustalX when the two sequences are in best alignment according to the alignment algorithm.

As used herein, the term “hybridization” or “hybridizes” under certain conditions is intended to describe conditions for hybridization and washes under which polynucleotide sequences that are significantly identical or homologous to each other remain bound to each other. Appropriate hybridization conditions can be selected by those skilled in the art with minimal experimentation as exemplified in Ausubel, F. A., et al., eds., Current Protocols in Molecular Biology Vol. 2, John Wiley and Sons, Inc., New York (1995). Additionally, stringency conditions are described in Sambrook et al. Molecular Cloning: A Laboratory Manual, 2nd ed., Cold Spring Harbor Laboratory Press, New York (1989). Variations on the conditions for low, moderate, and high stringency are well known in the art and may be used with the current invention.

As used herein, “antisense” refers to single, double or triple stranded polynucleotides and peptide nucleic acids (PNAs) that bind RNA transcript or DNA. Oligonucleotides derived from the transcription initiation site of the gene, e.g., between positions −10 and +10 from the start site, are a particular example. Triplex forming antisense can bind to double strand DNA thereby inhibiting transcription of the gene. Antisense molecules are typically 100% complementary to the sense strand but may be “partially” complementary in which only some of the nucleotides bind to the sense molecule (less than 100% complementary, e.g., 95%, 90%, 80%, 70% and sometimes less). Antisense molecules include and may be produced by methods including transcription from a gene or chemically synthesized (e.g., solid phase phosphoramidite synthesis). Antisense polynucleotides may be modified in order to provide resistance to degradation when administered to a patient. Particular examples include 5′ and 3′ linkages that are resistant to endonucleases and exonucleases present in various tissues or fluids in the body of an animal. Antisense polynucleotides do not require expression control elements to function in vivo. Such antisense molecules can be absorbed by the cell or enter the cell via passive diffusion. Antisense may also be introduced into a cell using a vector, such as a virus vector. However, antisense may be encoded by a nucleic acid so that it is transcribed, and, further, such a nucleic acid encoding an antisense may be operatively linked to an expression control element for sustained or increased expression of the encoded antisense in cells or in vivo.

Antisense polynucleotides may include L- or D-forms and additionally may be modified in order to provide resistance to degradation when administered to a patient. Particular examples include 5′ and 3′ linkages that are resistant to endonucleases and exonucleases present in various tissues or fluids in the body of an animal.

Antisense polynucleotides, to decrease expression of TNAP do not require expression control elements to function in vivo. Such antisense molecules can be absorbed by the cell or enter the cell via passive diffusion. Antisense may also be introduced into a cell using a vector, such as a virus vector. However, antisense may be encoded by a nucleic acid so that it is transcribed, and, further, such a nucleic acid encoding an antisense may be operatively linked to an expression control element for sustained or increased expression of the encoded antisense in cells or in vivo.

The term “detectable label” refers to any moiety that can be selectively detected in a screening assay. Examples include without limitation, radiolabels, (e.g., .sup.3H, .sup.14C, .sup.35S, .sup.1251, .sup.131I), affinity tags (e.g. biotin/avidin or streptavidin, binding sites for antibodies, metal binding domains, epitope tags, FLASH binding domains—See U.S. Pat. Nos. 6,451,569; 6,054,271; 6,008,378 and 5,932,474—glutathione or maltose binding domains) fluorescent or luminescent moieties (e.g. fluorescein and derivatives, GFP, rhodamine and derivatives, lanthanides etc.), and enzymatic moieties (e.g. horseradish peroxidase, .beta.-galactosidase, .beta.-lactamase, luciferase, alkaline phosphatase). Such detectable labels can be formed in situ, for example, through use of an unlabeled primary antibody which can be detected by a secondary antibody having an attached detectable label.

As used herein, the term “functionally expressed” refers to a coding sequence which is transcribed, translated, post-translationally modified (if relevant), and positioned in a cell such that the protein provides the desired function. With reference to a reporter cassette, functional expression generally means production of a sufficient amount of the encoded cell surface reporter protein to provide a statistically significant detectable signal to report transcriptional effects of a reporter polynucleotide.

“Naturally fluorescent protein” refers to proteins capable of forming a highly fluorescent, intrinsic chromophore either through the cyclization and oxidation of internal amino acids within the protein or via the enzymatic addition of a fluorescent co-factor. Typically such chromophores can be spectrally resolved from weakly fluorescent amino acids such as tryptophan and tyrosine. Endogenously fluorescent proteins have been isolated and cloned from a number of marine species including the sea pansies Renilla reniformis, R. kollikeri and R. mullerei and from the sea pens Ptilosarcus, Stylatula and Acanthoptilum, as well as from the Pacific Northwest jellyfish, Aequorea victoria; Szent-Gyorgyi et al. (SPIE conference 1999), D. C. Prasher et al., Gene, 111:229-233 (1992) and red and yellow fluorescent proteins from coral. A variety of mutants of the GFP from Aequorea victoria have been created that have distinct spectral properties, improved brightness and enhanced expression and folding in mammalian cells compared to the native GFP, (Green Fluorescent Proteins, Chapter 2, pages 19 to 47, edited Sullivan and Kay, Academic Press, U.S. Pat. No. 5,625,048 to Tsien et al., issued Apr. 29, 1997; U.S. Pat. No. 5,777,079 to Tsien et al., issued Jul. 7, 1998; and U.S. Pat. No. 5,804,387 to Cormack et al., issued Sep. 8, 1998). In many cases these functional engineered fluorescent proteins have superior spectral properties to wild-type proteins and are preferred for use as reporter genes in the present invention. Preferred naturally fluorescent proteins include without limitation, EGFP, YFP, Renilla GFP and DS red.

As used herein, the term “operably linked” refers to a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the DNA sequences being linked are typically contiguous and, where necessary to join two protein coding regions, are both contiguous and in reading frame. However, since enhancers generally function when separated from the promoter by several kilobases and intronic sequences may be of variable lengths, some polynucleotide elements may be operably linked but not contiguous. A structural gene (e.g., a HSV tk gene) which is operably linked to a polynucleotide sequence corresponding to a transcriptional regulatory sequence of an endogenous gene is generally expressed in substantially the same temporal and apoptosis-specific pattern as is the naturally-occurring gene.

The term “vector” refers to a plasmid, virus or other vehicle known in the art that can be manipulated by insertion or incorporation of a polynucleotide. Such vectors can be used for genetic manipulation (i.e., “cloning vectors”) or can be used to transcribe or translate the inserted polynucleotide (i.e., “expression vectors”). A vector generally contains at least an origin of replication for propagation in a cell and a promoter. Control elements, including expression control elements as set forth herein, present within a vector are included to facilitate transcription and translation (e.g., splicing signal for introns, maintenance of the correct reading frame of the gene to permit in-frame translation of mRNA and, stop codons etc.).

The term “expression control element” refers to one or more nucleic acid sequence elements that regulate or influence expression of a nucleic acid sequence to which it is operatively linked. An expression control element operatively linked to a nucleic acid sequence controls transcription and, as appropriate, translation of the nucleic acid sequence. An expression control element can include, as appropriate, promoters, enhancers, transcription terminators, gene silencers, a start codon (e.g., ATG) in front of a protein-encoding gene, etc.

A “promoter” is a minimal sequence sufficient to direct transcription. Although generally located 5′ of the coding sequence, they can be located in introns or 3′ of the coding sequence. Both constitutive and inducible promoters are included in the invention (see e.g., Bitter et al., Methods in Enzymology, 153:516-544 (1987)). Inducible promoters are activated by external signals or agents. Repressible promoters are inactivated by external signals or agents. Derepressible promoters are normally inactive in the presence of an external signal but are activated by removal of the external signal or agent. Promoter elements sufficient to render gene expression controllable for specific cell-types, tissues or physiological conditions (e.g., heat shock, glucose starvation) are also included within the meaning of this term.

For mammalian cell expression, constitutive promoters such as SV40, RSV and the like or inducible or tissue specific promoters derived from the genome of mammalian cells (e.g., metallothionein promoter) or from mammalian viruses (e.g., the mouse mammary tumor virus long terminal repeat; the adenovirus late promoter) or osteoclasts (e.g., Cbfa1, collagen I or ostecalcin gene promoter) may be used. Promoters produced by recombinant DNA or synthetic techniques may also be used to provide for transcription of antisense. Mammalian expression systems that utilize recombinant viruses or viral elements to direct expression may be engineered, if desired. For example, when using adenovirus expression vectors, the sequence coding for antisense may be ligated to an adenovirus transcription/translation control complex, e.g., the late promoter and tripartite leader sequence. (see e.g., Mackett et al., Proc. Natl. Acad. Sci. USA, 79:7415(1982); Mackett et al., J. Virol., 49:857(1984); and Panicali et al., Proc. Natl. Acad. Sci. USA, 79:4927(1982)).

Vectors based on bovine papilloma virus (BPV) have the ability to replicate as extrachromosomal elements (Sarver et al., Mol. Cell. Biol., 1:486 (1981)). Shortly after entry of an extrachromosomal vector into mouse cells, the vector replicates to about 100 to 200 copies per cell. Because transcription does not require integration of the plasmid into the host's chromosome, a high level of expression occurs. Alternatively, the retroviral genome can be modified for use as a vector capable of introducing and directing the expression of the gene in host cells (Cone et al., Proc. Natl. Acad. Sci. USA, 81:6349(1984)).

These vectors can be used for stable expression by including a selectable marker in the plasmid. A number of selection systems may be used to identify or select for transformed host cells, including, but not limited to the herpes simplex virus thymidine kinase gene (Wigler et al., Cell, 11:223(1977)), hypoxanthine-guanine phosphoribosyltransferase gene (Szybalska et al., Proc. Natl. Acad. Sci. USA, 48:2026(1962)), and the adenine phosphoribosyltransferase (Lowy et al., Cell, 22:817(1980)) genes can be employed in tk-, hgprt_ or aprt_ cells respectively. Additionally, antimetabolite resistance can be used as the basis of selection for dhfr, which confers resistance to methotrexate (Wigler et al., Proc. Natl. Acad. Sci. USA, 77:3567 (1980); O'Hare et al., Proc. Natl. Acad. Sci. USA, 78:1527(1981)); the gpt gene, which confers resistance to mycophenolic acid (Mulligan et al., Proc. Natl. Acad. Sci. USA, 78:2072(1981); the neomycin gene, which confers resistance to the aminoglycoside G-418 (Colberre-Garapin et al., J. Mol. Biol., 150:1(1981)); and the hygromycin gene, which confers resistance to hygromycin (Santerre et al., Gene, 30:147(1984)).

Mammalian expression systems further include vectors specifically designed for in vivo applications. Such systems include adenoviral vectors (U.S. Pat. Nos. 5,700,470 and 5,731,172), adeno-associated vectors (U.S. Pat. Nos. 5,354,678, 5,604,090, 5,780,447), herpes simplex virus vectors (U.S. Pat. No. 5,501,979) and retroviral vectors (U.S. Pat. Nos. 5,624,820, 5,693,508 and 5,674,703 and WIPO publications WO92/05266 and WO92/14829). Bovine papilloma virus (BPV) has also been employed in gene therapy (U.S. Pat. No. 5,719,054). Such vectors also include CMV based vectors (U.S. Pat. No. 5,561,063). In addition to viral vectors suitable for expression in vivo, lipids for intracellular delivery of polypeptides (including antibodies) and polynucleotides (including antisense) also are contemplated (U.S. Pat. Nos. 5,459,127 and 5,827,703). Combinations of lipids and adeno-associated viral material also can be used for in vivo delivery (U.S. Pat. No. 5,834,441).

Since the list of technical and scientific terms cannot be all encompassing, any undefined terms shall be construed to have the same meaning as is commonly understood by one of skill in the art to which this invention belongs.

The current invention provides material and methods directed towards the therapeutic benefits of modulating TNAP activity to in turn effect mineral deposition. TNAP mediated mineral deposition is found in numerous tissues, and is implicated with arterial calcification and other pathological conditions. For example, the inhibition of TNAP activity at the site of arterial calcification is desirable as a means for increasing the local concentration of PPi. Increased PPi will antagonize the deposition of hydroxyapatite while simultaneously upregulating OPN expression by VSMCs and thus further contributing to reducing ectopic hydroxyapatite deposition. Thus, the specific pharmacological ablation of TNAP activity results in the amelioration/prevention of arterial calcification.

TNAP, as with all mammalian APs, is inhibited uncompetitively by a number of inhibitors that include L-homoarginine (Fishman et al., 1971), as well as some non-related compounds, such as levamisole (Van Belle, 1976) and theophylline (Farley et al., 1980). However, these known inhibitors of TNAP are not entirely specific for this AP isozyme and may have low affinity, requiring the in vivo administration of very high concentrations to achieve biological effects. Thus, provided herein are novel structures that can either be used directly as specific modulators or at least will be suitable scaffolds to enable turning weaker binders into potent and selective modulators. There is additionally provided screening methods for modulators of TNAP activity.

According to one embodiment of the invention, an agent is administered to a subject that modulates TNAP activity, whether by modulating TNAP enzyme activity or modulating TNAP expression (either transciptionally or post-translationally). For example, TNAP activity can be inhibited or reduced by administering an agent comprising a small molecule (e.g., Compound ID: 5361418, Compound ID: 5804079, Compound ID: 5923412, dexamisole, D-tetramisole, forphenicine, L-homoarginine, L-tetramisole, Levamisole, or theophylline), an antisense, or an antibody specific for TNAP.

### EXAMPLES

The pharmacological ablation of TNAP activity leads to an increase in the concentrations of extracellular PPi that will result in amelioration/prevention of arterial calcification. Since arterial calcification is a condition associated with the development of atherosclerosis, we will test this hypothesis using the NPP1-deficient model of osteoarthritis and arterial calcification described above, but also the Apolipoprotein E-deficient mouse model that mimics the development of atherosclerotic plaques as seen in humans. As a first step toward targeting TNAP therapeutically, we have optimized a microtiter plate enzymatic assay using p-nitrophenylphosphate as substrate and measuring liberated p-nitrophenol as product, confirming suitable assay performance for the high-throughput environment. We have performed numerous HTS assays on chemical libraries containing 53,080 compounds and have succeeded in identifying small molecule, drug-like lead compounds that can be further modified to obtain highly specific TNAP inhibitors for in vivo use. Initial hits from this screen were counter-screened using very similar assay condition but using PPi as substrate and using the Biomol reagent to detect liberated phosphate as product. Subsequent tests will also be run using both assay designs to select those compounds that inhibit TNAP but not other related human phosphatases or NPP1. We use a combination of computer modeling, docking experiments and chemical synthesis to further modify the HTS hits to design novel, improved inhibitors of TNAP activity for use as in vivo therapeutics and to test these novel TNAP inhibitors in vivo for their ability to ameliorate/prevent arterial calcification in our animal models of arterial calcification and atherosclerosis.

### EXAMPLE 1

### TNAP Insufficiency Leads to Osteomalacia Due to an Arrest in the Propagation of Hydroxyapatite Crystals Outside the Matrix Vesicles

Mice lacking a functional Akp2 gene represent a model of infantile hypophosphatasia (Fedde et al., 1999). The animals display elevated plasma levels of known substrates of TNAP, i.e., inorganic phosphohate (PPi) and pyridoxal-5′-phosphate (PLP, a hydrophilic form of Vitamin B6) and develop impaired bone mineralization 6 to 10 days after birth and die at around 12 to 14 days of age. The mice also developed extensive epileptic seizures and suffer from apnea, increased apoptosis in the thymus and abnormal lumbar nerve roots (Narisawa et al., 1997). The administration of pyridoxal, a hydrophobic form of Vitamin B6 that can easily traverse biological membranes, suppresses the epileptic seizures and reverses apoptosis in the thymus and the lumbar nerve roots (Narisawa et al., 2001). However, hypomineralization and accumulation of osteiod continue to worsen with age and even these pyridoxal-treated mice are unable to live beyond 25 days. So abnormalities in the metabolism of PLP lead to Vitamin B6 deficiency in peripheral tissues and explain many of the abnormalities of infantile hyphosphatasia, but are not the basis for abnormal mineralization that characterizes this disease.

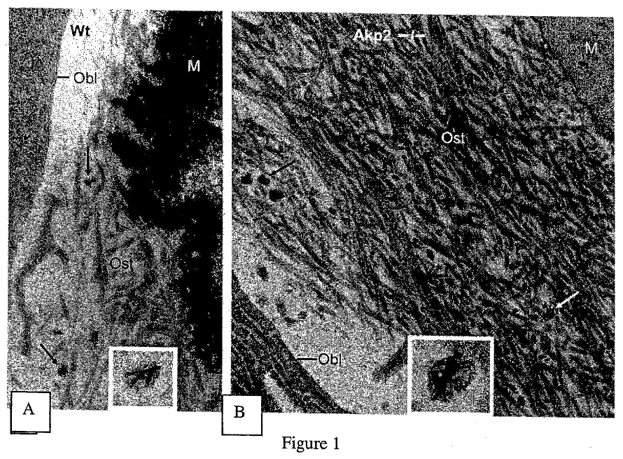

Deposition of hydroxyapatite during bone mineralization initiates within the lumen of membrane-limited MVs. Therefore, this example details the characterization of the ultrastructural localization, the relative amount and ultrastructural morphology of bone mineral in the tibial growth plates and in subadjacent metaphyseal bone in the Akp2−/− mice (Anderson et al., 2004). Alizarin red staining, micro-Computed Tomography, and Fourier Transform Imaging Spectroscopy confirm a significant overall decrease of mineral density in the cartilage and bone matrix of Akp2−/− mice. High-resolution transmission electron microscopy indicated that mineral crystals were initiated within MVs of the growth plate and bone of TNAP-deficient mice (FIG. 1). However, mineral crystal proliferation and growth was inhibited in the matrix surrounding MVs, as is the case in the human disease hypophosphatasia. These data suggested that hypomineralization in TNAP-deficient mice results primarily from an inability of mineral crystals within MVs to self-nucleate and to proliferate beyond the protective confines of the MV membranes. This failure of the second stage of mineral formation is caused by an excess of the mineral inhibitor PPi in the extracellular fluid around MVs.

### EXAMPLE 2

### Enpp1 Knockout Mice and Ank/Ank Mutant Mice are Models of Ankylosis and Osteoarthritis

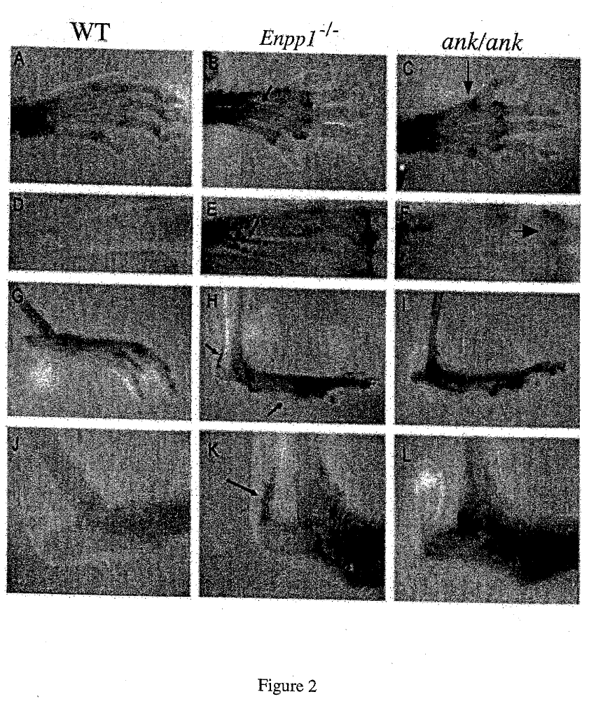

Mice deficient in NNP1 or defective in the PPi channeling function of ANK (ank/ank) have decreased levels of extracellular PPi and display soft-tissue ossification. Enpp1−/− mice develop features essentially identical to the previously described phenotype of the tiptoe walking mice (Okawa et al., 1998). These include the development of hyperostosis, starting at approximately three weeks of age, in a progressive process that culminates in ossific intervertebral fusion and peripheral joint ankylosis, as well as Achilles tendon calcification. The ank/ank mice have also been characterized as model of ankylosis (Ho et al., 2000). However, examination of whole mounts of their alizarin red stained skeletons consistently revealed subtle differences in the phenotypes of these mice (Harmey et al., 2004). It appears that the Enpp1−/− mice have a more severe soft tissue ossification phenotype than the ank/ank mutant mice. (FIG. 2). Enpp1−/− mice display soft tissue mineralization in the metatarsal bones (FIGS. 1B and 1E) as well as ossification of the Achilles tendon (FIG. 1H). Whereas the ank/ank mutants, though clearly displaying ectopic calcification (FIG. 1C), do not have the same degree of ossification as observed in Enpp1−/− mice (FIGS. 1C, 1F, 1I and 1L).

To determine the differences between NPP1 and ANK, we crossbred Enpp1−/− and ank/ank mice. It has been determined that these molecules act on separate pathways because the Enpp1−/−; ank/ank double deficient mice have greater degree of soft tissue ossification than do the single mutant animals. Specifically, Enpp1−/−; ank/ank double-deficient mice displayed a greater degree of perispinal ligament ossification than the single-deficient mice as determined by von Kossa staining of the spines (Harmey et al., 2004). Therefore, NPP1 and ANK have distinct effects on extracellular PPi concentrations. This was confirmed by examination of ANK and NPP1 localization in osteoblasts and MVs. Western blot analysis of ANK and NPP1 localization has revealed that both are present in osteoblast but only NPP1 is present in MVs. The absence of ANK in MVs suggests that its PPi-channeling function is not required for initiation and propagation of hydroxyapatite crystals and that is TNAP and NPP1 that are responsible for this process. Therefore, the absence of NPP1 in the MVs of Enpp1−/− osteoblasts results in a greater deficit in PPi levels than an absence of ANK. The deficit in extracellular PPi production in ank/ank mice results only from the decreased activity of ANK in the osteoblasts. Presumably there is still a sufficient amount of PPi within the MVs of the ank/ank mice such that the phenotypic abnormalities are not as severe as in Enpp1−/− mice.

### EXAMPLE 3

### Crossbreeding of Enpp1−/− or Ank/Ank mice to Akp2−/− Mice Corrects the Extracellular PPi and Osteopontin Levels and Ameliorates Their Respective Bone Abnormalities

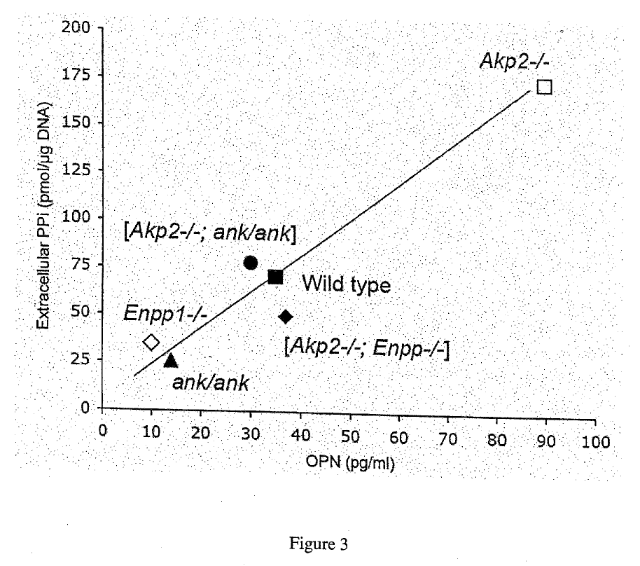

To determine whether affecting the function of either NPP1 or ANK would have beneficial consequences on hypophosphatasia by reducing the amounts of extracellular PPi in the Akp2−/− mice, we bred either Enpp1−/− or ank/ank mice to the Akp2−/− mice. A normalization of extracellular PPi levels as well as an improvement of the abnormalities in these Akp2−/− mice was observed. It was additionally observed that the life span of these mice doubled from about 12 days to 25 days (Hessle et al., 2002; Harmey et al., 2004). Moreover, skeletal correction was site-specific. The hypomineralization in the calvaria and vertebral apophyses was corrected but not of the appendicular (Anderson et al., 2005). Yet, expression of another mineralization inhibitor, OPN, was decreased in both the Enpp1−/− and the ank/ank osteoblasts (Johnson et al., 2003). RT-PCR analysis of wild-type osteoblasts treated with exogenous PPi revealed an increase in OPN expression and decreased NPP1 and ANK expression. This supports a direct regulation of OPN expression by NPP1 and ANK expression, mediated by PPi. Akp2−/− mice demonstrate significant elevations in serum OPN levels may also be altered to wild-type mice, as measured by ELISA. Both PPi and OPN levels are normalized in Akp2−/−; Enpp1−/− and Akp2−/−; ank/ank double mutant mice and that these parameters are in clear correlation (FIG. 3). Therefore, under normal conditions the concerted action of TNAP, NPP1, and ANK regulate PPi and OPN levels and therefore control hydroxyapatite deposition outside of the MVs. Hypophosphatasia arises from deficits in TNAP activity, resulting in an increase in PPi levels and concomitant increase in OPN levels. The combined inhibitory effect of these molecules leads to hypomineralization.

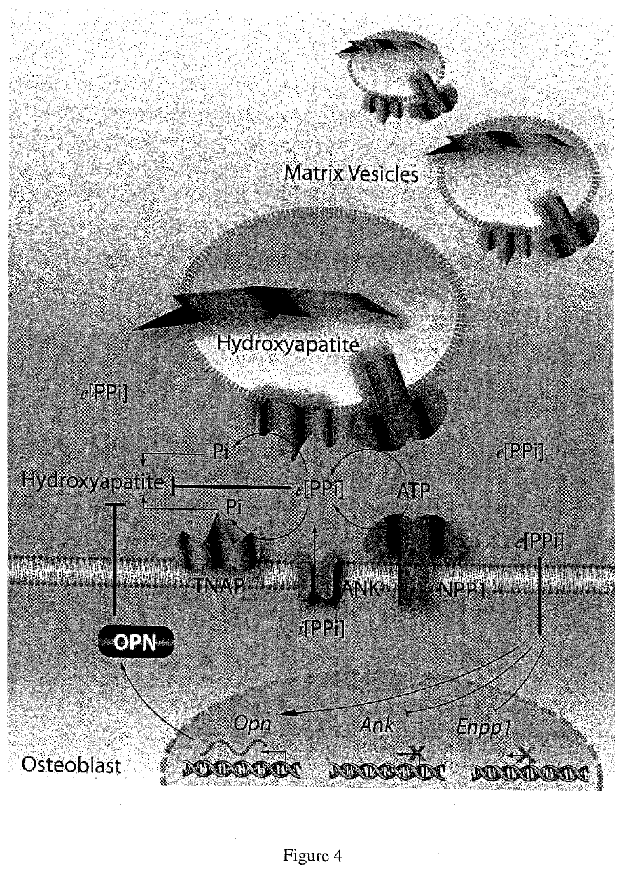

The elucidated mechanisms regulating extracellular PPi concentration is presented in FIG. 4. Hypophosphatasia in the Akp2−/− mice arises from deficits in TNAP activity, resulting in an increase in extracellular PPi levels and a concomitant increase in OPN levels. The combined inhibitory effect of these molecules leads to hypophosphatasia. Whereas, an NPP1 or ANK deficiency leads to a decrease in the extracellular PPi and OPN pools, thereby enabling ectopic soft tissue ossification. The hypomineralization defects in Akp2−/− mice, along with elevated PPi and OPN levels are normalized by ablation of either the NPP1 or ANK gene. Conversely, ablating the function of TNAP causes normalization of the abnormalities in the Enpp1−/− and ank/ank mutant mice via resulting increase in the concentrations of two inhibitors of mineralization, i.e., extracellular PPi and OPN.

### EXAMPLE 4

### Enpp1−/− and Ank/Ank Mice as Models of Vascular Calcification

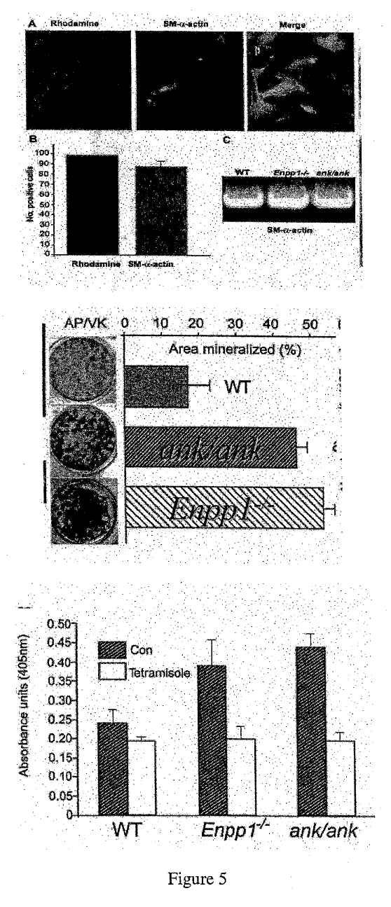

Given the coordinated function of NPP1 and ANK on extracellular PPi concentrations and the similarity in the bone abnormalities found in the Enpp1−/− and the ank/ank mutant mice, it is expected that the similarities will also extend to vascular calcification sites. Our data show that ank/ank mutant mice display signs of arterial calcification just as Enpp1−/− mice do. Using a collagenase digestion method, we isolated VSMCs and identified them as such by immunofluorescence and RT-PCR detection of smooth muscle alpha-actin (SMAA), and obtained a population of cells in which, on average, 89% of cells stained positive for SMAA (FIG. 5). Using these VSMC cultures, we established that wild-type VSMCs do indeed possess TNAP activity; secondly that VSMCs when cultured in the presence of .beta.-glycerophosphate can lay down mineral in a manner and with kinetics similar to osteoblastic cultures; thirdly, that VSMCs from Enpp1−/− and ank/ank mutant mice display higher TNAP activity than WT cells and that they are produce significantly more mineral when cultured in this system thereby strengthening the use of Enpp1−/− and ank/ank mice as models for vascular calcification. We have shown that tetramisole treatment of Enpp1−/− and ank/ank derived VSMC in vitro inhibits TNAP activity (FIG. 5).

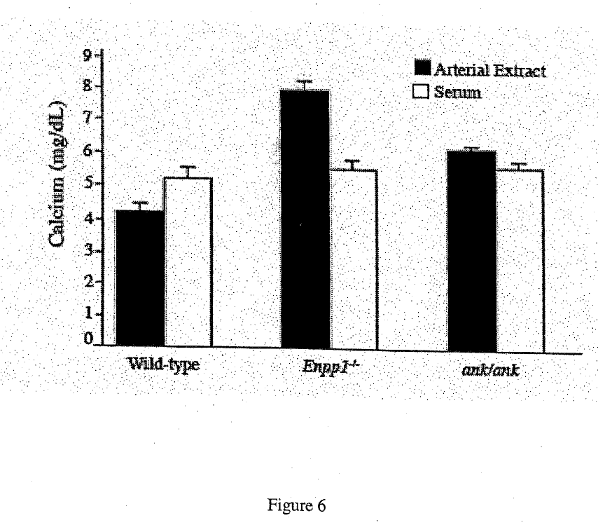

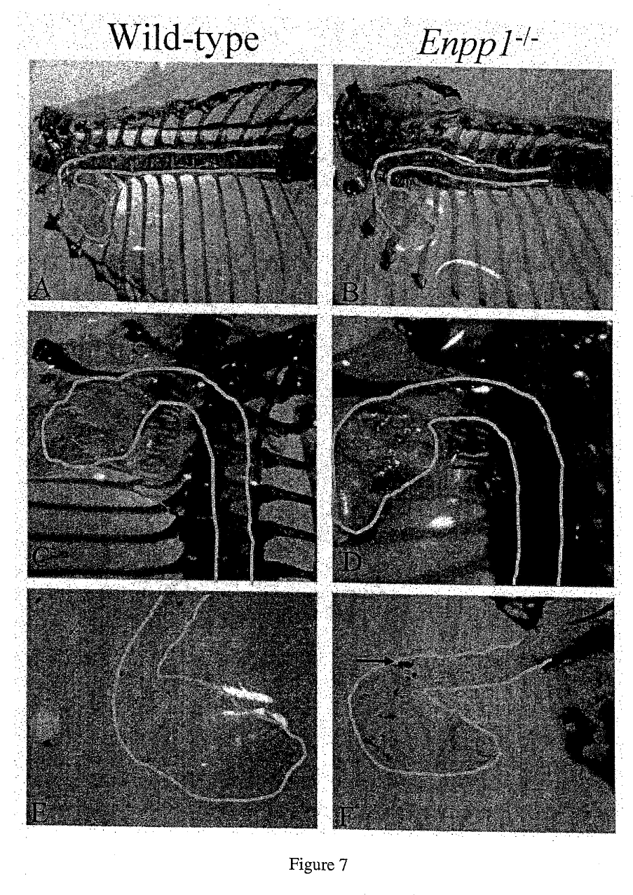

The amount of calcium deposited in wild-type, Enpp1−/−, and ank/ank aortas was quantified and is presented in FIG. 6. Mice at three months of age show a higher degree of calcification in Enpp1−/− and ank/ank compared to wild-type control animals. Moreover, there is more calcification in Enpp1−/− mice than in ank/ank mice. Whole mount preparations of heart and aorta from wt and Enpp1−/− mice were stained with Alizarin red to visualize calcium deposition. In FIG. 7 multiple foci are present representing aortic calcification in Enpp1−/− mice, while none are present in the control mice. The digital outline of the heart and aorta is provided in FIG. 7 for clarity.

### EXAMPLE 5

### Inhibition of TNAP Activity In Vivo Leads to Improvement in Spine Flexibility in Enpp1−/− and Ank/Ank Mutant Mice

Thus, deletion of the Akp2 gene results in elevation of PPi and OPN concentrations in bone matrix and suppresses soft tissue ossification in both Enpp1−/− and ank/ank mice. We have, therefore, determined that the chemical ablation of TNAP activity will provide a treatment for pathological conditions caused by decreased levels of extracellular PPi. To select an inhibitor of TNAP for in vivo use, the efficiency by which L-homoarginine, theophylline and tetramisole were able to suppress TNAP activity, elevate extracellular PPi levels, and inhibit mineralization in the MC3T3-E1 osteoblastic cell line was measured.

Enpp1−/− and ank/ank mice were subcutaneously treated with tetramisole to inhibit soft-tissue mineralization and consequently improve the flexibility of their joints. Tetramisole was delivered via ALZET osmotic pumps (Durect Corporation, Cupertino, Calif.) at a dose of 10 μg/g/day. Treated mice of both genetic deficiencies showed an enhanced flexibility of the spine compared to those of control mice. (FIG. 10) Despite the fact that untreated Enpp1−/− mice are less flexible than untreated ank/ank mice, they did show a greater degree of improvement in response to treatment of tetramisole. These results suggest that treatment of soft-tissue ossification, including vascular calcification, by targeting the enzymatic activity of TNAP is effective for treating pathological conditions resulting from a reduced level of PPi in the extracellular space.

### EXAMPLE 7

### Site-Directed Mutagenesis and Computer Modeling Reveals which Residues Determine the Binding Specificity of TNAP Inhibitors

Mutagenesis study of structure-function relationships in alkaline phosphatases including the features responsible for the specific properties of alkaline phosphatase isozymes has been accelerated by the elucidation of the 1.8 Å structure of human placental alkaline phosphatase (PLAP) (Le du et al., 2001). Residue Y367 is a relevant feature of alkaline phosphatases. This residue is part of the subunit interface in PLAP dimers, where it protrudes from one subunit and is positioned within 5.6 Å of the catalytic Zn1 ion in the active site of the other subunit. Given the location of Y367 and its conservation in all mammalian alkaline phosphatases, we were able to predict that Y367 helps stabilize the uncompetitive inhibitors, L-Phe and L-Leu. Substitutions Y367A and Y367F significantly compromised the heat stability of the mutant PLAP enzymes, and had a profound destabilizing effect in the inhibition of L-Phe and L-Leu. We were also able to determine that the side chains of Tyr367, Phe1077, Gln108 and Asp91 form the pocket that accommodates L-Phe and L-Leu (Kozlenkov et al., 2002).

BLAST sequence alignment and the MODELLER program to compare the overall structures of GCAP, IAP, and TNAP provides information as to what stabilizes monomers in an alkaline phosphatase dimer and identifies fingerprints of the active cleft site characteristic of each alkaline phosphatase isozyme (for TNAP the active cleft site is referred to herein as TNAP modulation domain). It is determined by this analysis of the active pocket of TNAP that the crystal structure of PLAP forms a ternary complex with the inhibitor L-Phe and phosphate covalently bound to the active site Ser92. This 1.6 Å resolution structure provides a solid foundation for structure-based compound design methods as provided herein.

The polynucleotide structure for TNAP is SEQ ID NO: 1 and can be found as accession number P05186, incorporated herein by reference. The corresponding polynucleotide sequence is SEQ ID NO: 2 and is found as accession number NM000478, incorporated herein by reference. It is well known that human TNAP comprises a seventeen (17) amino acid residue signal sequence that is cut off by the golgi during processing to create the mature polypeptide. That signal sequence is residues 1-17 of SEQ ID NO: 1. In this application the residues of the TNAP polypeptide are numbered according to the mature polypeptide, (i.e., post cleavage of the signal sequence), which can be found at SEQ ID NO: 3.

Several isozyme-specific inhibitors of alkaline phosphatases have been reported. They include L-amino-acids, such as L-phenylalanine, L-tryptophan, L-Leucine, L-homoarginine (Fishman & Sie, 1971; Doellgast & Fishman, 1977) as well as some non-related compounds, such as levamisole, the L-stereoisomer of tetramisole (Van Belle, 1976) and theophylline (Farley et al., 1980). The inhibition is of a rare uncompetitive type and while the biological implications of his inhibition are unknown, the inhibitors have proven to be useful in the differential determination of alkaline phosphatase in clinical chemistry (Fishman, 1974; Mulivor et al, 1978).

To determine which amino acid residues are responsible for the marked differences in inhibition selectivity, we produced mutants of both TNAP and PLAP isozymes, concentrating on those residues that are different in the active site area. The TNAP mutant were generated using either a QuikChange XL kit according to manufacturer's mutagenesis protocol, (Stratagene, La Jolla, Calif.) of by employing an overlap extension method. Mutations were confirmed by ligation of the product into pCRII/TOPO (Invitrogen, Carlsbad, Calif.) and sequencing. (See Kozlenkov A, et al., 2002). The mutant constructs (TNAP-Flag/pcDNA3) were transfected into COS-7 cells using Superfect (Quiagen, Valencis, Calif.) for transient expression. Secreted proteins were collected from the media between 3 and 6 days post transfection and purified using affinity chromatography with an anti-FLAG antibody gel (Sigma, St. Louis, Mo.) according to manufacturer's protocol.

Relative specific activities of the mutants were measured as described in Kozlenkov A, et al., 2002. In brief, samples of the enzymes were added to microtiter plates coated with M2 anti-FLAG antibody, and saturating activities with the substrate pNPP were measured in 1 M DEA/HCl buffer, pH 9.8, containing 1 mM MgCl.sub.2 and 20 .micro.M ZnCl.sub.2. The determinations of Km and the inhibition studies were done in the same buffer, with varying concentrations of pNPP and/or inhibitors. Levamisole (L-tetramisole), L-homoarginine, L-phenylalanine (all from Sigma) and theophylline (Fluka) were used as reagents in the inhibition studies. Ki values for the uncompetitive inhibitors were obtained from the inhibition studies using 20 mM pNPP (saturating substrate concentration) as well as at 1 mM pNPP. The results of enzyme kinetics studies were analyzed by nonlinear regression using software Prism 3.02 (GraphPad Software). Variations in Ki within a factor of 2 were not considered functionally relevant.

TNAP and PLAP structures were superimposed and the amino acid differences within a 12 Å radius around the catalytic Zn1 ion were pinpointed. The difference between human TNAP and chicken TNAP were also mapped because it is reported that chicken TNAP is much less susceptible to levamisol than is human TNAP. In total, six positions with amino acid differences were found, which could be clustered into two groups. The first group, using the TNAP numbers of SEQ ID NO: 3, includes residues 433 and 434, and the second group includes residues 108, 109, 120, and 166. In addition, Asp168 and Tyr371 were also investigated.

The Kcat, Km for substrate and the Ki towards the uncompetitive inhibitors L-homoarginine, levamisole, theophylline, and L-phenylalanine were determined for all mutants and the results are presented in Table 1.

TABLE 1

| | | | Kᵢ for various inhibitors | | | | |
|---|---|---|---|---|---|---|---|
| | | | L-hArg | L-Phe | Levamisole | Theophylline | Theophylline |
| Mutants | k_{cat} (10⁵ × s⁻¹) | Kₘ (mM) | (mM) | (mM) | (μM) | (20)* (μM) | (1)* (μM) |
| hTNALP | 2.1 ± 0.2 | 0.36 ± 0.02 | 1.4 ± 0.1 | 19 ± 6 | 16 ± 1 | 25 ± 2 | 82 ± 11 |
| chTNALP | ND | 0.64 ± 0.03 | 1.9 ± 0.2 | 11 ± 0.9 | 136 ± 20 | 111 ± 8 | 393 ± 150 |
| hPLALP | 0.46 | 0.38 ± 0.02 | 59 ± 15 | 0.75 ± 0.05 | 563 ± 56 | 947 ± 120 | 932 ± 90 |

hTNALP

[symbol]

| | | | | | | | |
|---|---|---|---|---|---|---|---|
| A108 | 4.3 ± 0.1 | 0.28 ± 0.03 | 14 ± 1 | 27 ± 4 | 20 ± 1 | 15 ± 1 | 31 ± 3 |
| F108 | 1.8 ± 0.1 | 0.19 ± 0.01 | 48 ± 11 | 3.7 ± 0.5 | 19 ± 2 | 5.6 ± 0.3 | 16 ± 2 |
| A109 | 0.39 ± 0.02 | 0.27 ± 0.02 | 1.3 ± 0.2 | 20 ± 4 | 16 ± 1 | 29 ± 2 | 104 ± 10 |
| Q109 | 0.33 ± 0.05 | 0.30 ± 0.02 | 1.6 ± 0.1 | 4.0 ± 1.1 | 50 ± 4 | 40 ± 12 | 317 ± 33 |
| (F104, Q109) | 0.46 ± 0.03 | 0.19 ± 0.03 | 15 ± 2 | 2.1 ± 0.4 | 36 ± 3 | 10 ± 0.4 | 6 ± 1 |
| A120 | 2.0 ± 0.8 | 0.31 ± 0.02 | 1.4 ± 0.1 | 24 ± 2 | 14 ± 1 | 19 ± 1 | 103 ± 6 |
| D120 | 1.2 ± 0.1 | 0.37 ± 0.03 | 1.4 ± 0.1 | 34 ± 4 | 13 ± 1 | 24 ± 2 | 104 ± 10 |
| N120 | 2.9 ± 0.1 | 0.35 ± 0.01 | 1.3 ± 0.1 | 23 ± 8 | 14 ± 0.5 | 22 ± 1 | 83 ± 10 |
| A166 | 1.6 ± 0.1 | 0.28 ± 0.02 | 1.6 ± 0.3 | 24 ± 4 | 26 ± 1 | 33 ± 2 | 67 ± 4 |
| N166 | 3.4 ± 0.1 | 0.39 ± 0.05 | 1.8 ± 0.1 | 32 ± 5 | 15 ± 1 | 30 ± 2 | 87 ± 4 |
| A168 | 1.4 ± 0.3 | 0.29 ± 0.03 | 1.5 ± 0.3 | 30 ± 4 | 18 ± 2 | 25 ± 1 | 58 ± 6 |
| N168 | 0.59 ± 0.01 | 0.28 ± 0.03 | 1.8 ± 0.1 | 42 ± 6 | 20 ± 1 | 33 ± 2 | 66 ± 6 |
| A371 | 0.46 ± 0.03 | 0.29 ± 0.02 | 31 ± 3 | 65 ± 24 | 122 ± 6 | 52 ± 3 | 118 ± 11 |
| A433 | 0.68 ± 0.05 | 0.41 ± 0.01 | 1.6 ± 0.2 | 31 ± 3 | 18 ± 2 | 52 ± 4 | 134 ± 8 |
| D433 | 0.98 ± 0.05 | 0.52 ± 0.02 | 1.5 ± 0.2 | 23 ± 5 | 18 ± 1 | 51 ± 2 | 218 ± 28 |
| A434 | 2.8 ± 0.1 | 0.69 ± 0.03 | 1.5 ± 0.1 | 21 ± 6 | 133 ± 7 | 121 ± 9 | 219 ± 20 |
| Q434 | 2.5 ± 0.1 | 0.79 ± 0.10 | 2.0 ± 0.1 | 9.8 ± 1 | 268 ± 53 | 126 ± 27 | 374 ± 20 |
| G434 | 1.6 ± 0.1 | 0.50 ± 0.04 | 1.6 ± 0.2 | 9.6 ± 1 | 311 ± 38 | 158 ± 23 | 176 ± 20 |
| S434 | 2.3 ± 0.1 | 0.66 ± 0.03 | 1.6 ± 0.1 | 13 ± 2 | 163 ± 17 | 186 ± 10 | 248 ± 18 |
| B434 | 2.3 ± 0.1 | 2.0 ± 0.1 | 6.8 ± 0.5 | 43 ± 8 | 731 ± 70 | 1240 ± 80 | 2350 ± 310 |
| (D433, E434) | 1.4 ± 0.1 | 3.1 ± 0.9 | 13 ± 1.6 | 206 ± 111 | 1430 ± 120 | 2400 ± 250 | 5200 ± 750 |

hPLALP

[symbol]

| | | | | | | | |
|---|---|---|---|---|---|---|---|
| E107 | 0.43 ± 0.02 | 0.49 ± 0.03 | 5.7 ± 0.9 | 3.8 ± 0.4 | 1050 ± 40 | 4200 ± 260 | 5800 ± 670 |
| G108 | 0.57 ± 0.02 | 0.30 ± 0.01 | 60 ± 9 | 2.4 ± 0.3 | 458 ± 30 | 540 ± 85 | 650 ± 95 |
| (E107, G108) | 0.45 ± 0.02 | 0.38 ± 0.01 | 12 ± 1.6 | 23 ± 2 | 1150 ± 60 | 6400 ± 500 | 9700 ± 1300 |
| S119 | 0.6 ± 0.1 | 0.32 ± 0.05 | 61 ± 13 | 0.67 ± 0.08 | 630 ± 90 | 710 ± 70 | 920 ± 90 |
| D165 | 0.44 ± 0.03 | 0.33 ± 0.04 | 59 ± 25 | 0.54 ± 0.04 | 680 ± 140 | 690 ± 40 | 1080 ± 100 |
| R428 | 1.4 ± 0.3 | 0.6 ± 0.03 | 36 ± 4 | 0.54 ± 0.05 | 460 ± 40 | 330 ± 40 | 630 ± 50 |
| G429 | 0.36 ± 0.05 | 0.09 ± 0.01 | 12 ± 2 | 0.09 ± 0.01 | 474 ± 56 | 200 ± 40 | 216 ± 7 |
| H429 | 0.95 ± 0.04 | 0.14 ± 0.01 | 10.7 ± 1.5 | 0.31 ± 0.03 | 50 ± 3 | 34 ± 1 | 115 ± 8 |
| (R428, H429) | 1.4 ± 0.1 | 0.09 ± 0.006 | 14 ± 1 | 0.35 ± 0.04 | 34 ± 1 | 19 ± 1 | 76 ± 5 |
| (E107, G429) | 0.10 ± 0.01 | 0.11 ± 0.01 | 1.5 ± 0.2 | 1.0 ± 0.16 | 535 ± 80 | 161 ± 54 | 443 ± 70 |
| (G108, G429) | 0.44 ± 0.02 | 0.08 ± 0.01 | 18 ± 2 | 0.49 ± 0.06 | 480 ± 60 | 158 ± 15 | 189 ± 13 |

[symbol]

indicates data missing or illegible when filed

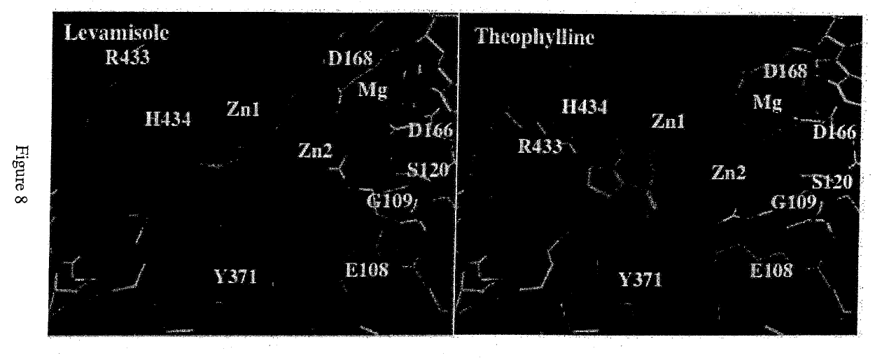

Thus, it is herein shown that the identity of residue 108 in TNAP largely determines the specificity of inhibition by L-homoarginine. The conserved Tyr-37 1 is also necessary for binding of L-homoarginine. In contrast, the binding of levamisole to TNAP is mostly dependent on His-434 and Tyr-371, but not residues 108 or 109. The main determinant of sensitivity to theophylline is His-434. This data together with ab initio docking of inhibitors in the active site of TNAP has allowed us to identify the binding conformation for these inhibitors in the active site of TNAP (FIG. 8).

Compounds identified by this method can then be synthesized, purchased or otherwise obtained, and can then be used as modulators or can be further screened for modulation activity against TNAP.

Exemplary compounds include Compound ID: 5361418, Compound ID: 5804079, Compound ID: 5923412, dexamisole, D-tetramisole, forphenicine, I-homoarginine, L-tetramisole, Levamisole, or theophylline, analogues thereof and derivatives thereof. The term “analogue” means a structurally similar molecule that has at least part of the function of the comparison molecule. In other words, the analogue would still retain at least a part of the modulation activity of the comparison molecule, i.e. an L-tetramisole analogue would retain at least a part of the TNAP inhibitory activity of L-tetramisole. As used herein, the term “derivative” means a modified form of the molecule, that is, the molecule is chemically or otherwise modified in comparison to the original form. Again, the derivative would still retain at least a part of the modulation activity of the unmodified molecule. For example, a derivative of a TNAP inhibitor would be a modified form of an antagonist molecule that inhibits, decreases, reduces or prevents TNAP expression or an activity.

### EXAMPLE 8

### Identification of Compounds that Modulate TNAP Activity Through Chemical Library Screening

Recombinant human FLAG-TNAP was expressed in COS-1 cells as previously described in Kozlenkov et al., 2004 and the collected supernatant containing the secreted enzyme was dialyzed against TBS containing 1 mM MgCl.sub.2 and 20 mM ZnCl.sub.2 to remove Pi from the serum free medium. Prior to the screening, the TNAP stock solution was diluted 1/120 times and 12.0 microliter of the diluted TNAP was dispensed into 96 well microtiter plates with area bottom from Costar (Corning, N.Y.) by an auto dispenser from Matrix (Hudson, N.H.). A robotic liquid handler, Biomek FX from Beckman Coulter (Fullerton, Calif.) dispensed 2.5 microliter of each compound from the library plates dissolved in 10% DMSO. The plates were incubated at room temperature for at lease one hour to allow TNAP to interact with each compound prior to addition of 10.5 microliter substrate stock solution (1.19 mM pNNP) to achieve a final volume of 25.5 microliter per well and a final substrate concentration of 0.5 mM. After thirty minutes incubation, OD405 was measured with a plate reader, Analyst HT from Molecular Devices (Sunnyvale, Calif.). Both TNAP and substrate solution were made in diethanolamine (DEA) buffer, pH 9.8 and the final reaction contains 1M DEA-1 mM MgCl.sub.2-20 mM ZnCl.sub.2. Under these conditions we achieved an OD 405 range (˜0.4) while maintaining an inhibition response of around 50% for levamisole and phosphate which can be used as positive controls during the screening.

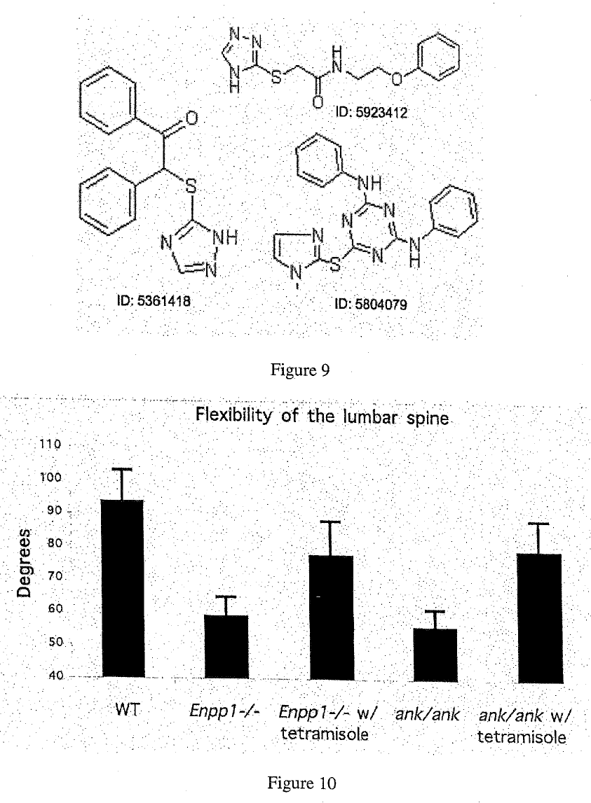

After screening 53,080 compounds, we have confirmed 4 compounds that inhibit the enzymatic activity of TNAP. One of the compounds turned out to levamisole. The other three compounds (FIG. 9) conform to Lipinski's rule of 5, i.e., molecular weight less than 500, less than 5 Hydrogen-bond donors, less than 5 Hydrogen-bond receptors, less than 10 rotational bonds and octanol/water repartition coefficent (LogP) <5.

Agents that are screened by this modulator screening method to determine their ability to modulate TNAP activity can include, but are not limited to, a peptide, polypeptide, peptidomimetic, non-peptidyl compound, carbohydrate, lipid, a synthetic compound, a natural product, an antibody or antibody fragment, a small organic molecule, a small inorganic molecule, and a nucleotide sequence. In one embodiment the screening method can be performed in vitro. Furthermore, the screening method can be performed as a High Throughput Screening assay (HTS). In an alternate embodiment, the screening method can be performed as a computational modeling study. In a still further embodiment, the screening method can be performed in vivo; for example employing animal models. Moreover, the screening method can be performed using transgenic cell lines. These various formats for performing the screening method of the current invention are not mutually exclusive, and as such can be used in combinations with one another.

### EXAMPLE 9

### Testing Known and Improved TNAP Inhibitors In Vitro and In Vivo for Their Ability to Ameliorate and Prevent Vascular Calcification in Mouse Models

Two genetically engineered mouse models, the low-density lipoprotein receptor knockout (Ldlr−/−) mouse and the apolipoprotein E knockout (ApoE−/−) mouse, are widely used as atherosclerotic models. (Ishabashi et al., 1993; Plump et al., 1992). Both these mouse models display unstable atherosclerotic plaques, including intra-plaque hemorrhage, vascular calcification, thinning of the fibrous cap, size of the necrotic core and macrophage content. However, the Ldlr−/− develop milder abnormalities that take longer to appear. Thus, for this current example, the ApoE−/− mouse model will be used to assess the effect of inhibiting TNAP activity in preventing arterial calcification and whether this results in an improvement in the atherosclerotic disease. The effect of inhibiting TNAP will be investigated using parameters critical to atherogenesis, such as lipid accumulation in the lesions, number of inflammatory cells, development of calcification and lesion morphology (i.e., collagen architecture and smooth muscle cell presence).

In one example of this pharmacological study tetramisole will be used. Tetramisole is known to normalize TNAP activity in Enpp1−/− and ank/ank VSMCs and is also known to raise PPi levels in MC3T3-E1 cells. Performing a dose response study (results not shown) it was determined that a dose of 30 micrograms/g BW is well tolerated for this compound. The dose is preferably delivered using an Alzet Osmotic Pump, thereby providing constant and controlled drug delivery rather than multiple injections. For ApoE−/− mice arterial calcification is established by 3-months of age. Thus, the pump can feasibly be planted as early as 1-month of age, allowing treatment to begin at least as early.

Initial treatment of the ApoE−/− mice with tetramisol will be for 28-days, after which blood will be collected. The PPi and OPN levels in these blood samples will be measured to thereby monitor drug action. Similarly, TNAP activity will be measured in serum. As such it can be established that a compound, in this case tetramisole, is modulating TNAP.

Histological analysis will be performed as well. Following treatment with a compound, test mice will be sacrificed and dissected. Aortic tissue will be collected as follows: perfusion with PBS followed by 10% Neutral Buffered Formalin via the left ventricle. The artery will be dissected, embedded in paraffin and serially sectioned (5 micrometers). To observe gross morphological changes Hematoxylin and Eosin staining will be performed on the aorta. Morphometric analysis will be performed on artery cross sections to determine difference in arterial wall thickness (intimal and medial areas). Perimeters of the lumen, the internal elastic lamina (IEL) and the external elastic lamina (EEL) will be obtained by tracing contours on a digitized image (aprox. 100 sections per artery). Results will be expressed as ratio of intimal area to medial wall area will give a quantitative measurement of the thickening of the arterial wall.

To identify vascular calcification phosphate deposition will be visualized by von Kossa staining (5% silver nitrate) using Nuclear Fast Red as a counter stain, and calcium will be detected using Alzerian Red S (0.5% pH 9.0) Elastic fiber will be visualized using the Verhoeff-Van Gieson method to reveal any changes in the arterial wall architecture of the treated mice. Oil Red O staining will be performed on ApoE−/− arterial sections to observe the effects of TNAP on lipid-containing atherosclerotic plaques. Moreover, arterial cross sections can be immunostained to determine upregulation of the adhesion molecules VCAM-1, MCP, and PAF, or upregulation of other molecules such as TNAP, OCN, OPN, MGP, BSP, BMP-2 and BMP-4.

Modulators identified by the modulator screening methods described herein are further studied according to this or a similar pharmacological study so that a pharmaceutical formulation comprising said modulators can be prepared and properly delivered as a treatment for the pathological conditions know or suspected to be treated by modulation of TNAP activity.

Various modifications and alterations of the invention will become apparent to those skilled in the art without departing from the spirit and scope of the invention, which is defined by the accompanying claims. For example, it should be noted that steps recited in any method claims below do not necessarily need to be performed in the order that they are recited. Those of ordinary skill in the art will recognize variations in performing the steps from the order in which they are recited. For example, in certain embodiments, steps may be performed simultaneously. The accompanying claims should be constructed with these principles in mind.

### REFERENCES

Farley, J. R., Ivey, J. L. and Baylink, D. J. (1980) Human skeletal alkaline phosphatase-Kinetic studies including pH dependence and inhibition by theophylline. J. Biol. Chem. 255: 4680-4686.

Fishman, W. H. and Sie, H.-G. (1971) Organ-specific inhibition of human alkaline phosphatase isoenzymes of liver, bone, intestine and placenta; L-phenylalanine, L-tryptophan and L-homoarginine. Enzymologia 41: 140-167.

Ho, A. M., Johnson, M. D., and Kingsley, D. M. (2000) Role of the mouse ank gene in control of tissue calcification and arthritis. Science 289:265-270.

Hessle, L., Johnson, K. A., Anderson, H. C., Narisawa, S., Sali, A., Goding, J. W., Terkeltaub, R., and Millán, J. L. (2002) Tissue-nonspecific alkaline phosphatase and plasma cell membrane glycoprotein-l are central antagonistic regulators of bone mineralization. Proc. Natl. Acad. Sci. USA 99:9445-9449.

Hsu, H. H. and Camacho, N. P. (1999) Isolation of calcifiable versicles from human atherosclerotic aortas. Atherosclerosis 143: 353-362

Hui, M., Li, S. Q., Holmyard, D., and Cheng, P. (1997) Stable transfection of nonosteogenic cell lines with tissue nonspecific alkaline phosphatase enhances mineral deposition both in the presence and absence of beta-glycerophosphate: possible role for alkaline phosphatase in pathological mineralization. Calcif Tissue Int 60:467-72.

Hui, M., and Tenenbaum, H. C. (1998) New face of an old enzyme: alkaline phosphatase may contribute to human tissue aging by inducing tissue hardening and calcification. Anat Rec 253:91-4.

Johnson, K. A., Hessle, L., Wennberg, C., Mauro, S., Narisawa, S., Goding, J., Sano, K., Millán, J. L. and Terkeltaub, R. (2000) Tissue-nonspecific alkaline phosphatase (TNAP) and plasma cell membrane glycoprotein-1 (PC-1) act as selective and mutual antagonists of mineralizing activity by murine osteoblasts. Am. J. Phys. Regulatory and Integrative Physiology 279: R1365-1377.

Johnson, K., Goding, J., Van Etten, D., Sali, A., Hu, S. I., Farley, D., Krug, H., Hessle, L., Millán, J. L., and Terkeltaub, R. (2003) Linked deficiencies in extracellular PP(i) and osteopontin mediate pathologic calcification associated with defective PC-1 and ANK expression. J. Bone. Min. Res. 18:994-1004.

Mathieu, P., Voisine. P., Pepin. A., Shetty, R., Savard. N., and Dagenais, F. (2005) Calcification of human valve interstitial cells is dependent on alkaline phosphatase activity. J Heart Valve Dis 14:353-7

Okawa, A., Nakamura, I., Goto, S., Moriya, H., Nakamura, Y., and Ikegawa, S. (1998) Mutation in Npps in a mouse model of ossification of the posterior longitudinal ligament of the spine. Nat Genet 19:271-273.

Rutsch, F., Vaingankar, S., Johnson, K., Goldfine, I., Maddux, B., Schauerte, P., Kalhoff, H., Sano, K., Boisvert, W. A., Superti-Furga, A., and Terkeltaub, R. (2001) PC-1 nucleoside triphosphate pyrophosphohydrolase deficiency in idiopathic infantile arterial calcification. Am. J. Pathol. 158:543-554.

Rutsch, F., Ruf, N., Vaingankar, S., Toliat, M. R., Suk A, Hohne, W., Schauer, G., Lehmann, M., Roscioli, T., Schnabel, D., Epplen, J. T., Knisely, A., Superti-Furga, A., McGill, J., Filippone, M., Sinaiko, A. R., Vallance, H., Hinrichs, B., Smith, W., Ferre, M., Terkeltaub, R., and Nurnberg, P. (2003) Mutations in ENPP1 are associated with ‘idiopathic’ infantile arterial calcification. Nat. Genet. 34:379-381.

Shioi, A., Nishizawa, Y., Jono, S., Koyama, H., Hosoi, M., and Morii, H. (1995) Beta-glycerophosphate accelerates calcification in cultured bovine vascular smooth muscle cells. Arterioscler Thromb Vasc Biol 15:2003-9.

Tanimura, A., McGregor, D. H., and Anderson, H. C. (1986a) Calcification in atherosclerosis. I. Human studies. J. Exp. Pathol. 2:261-273.

Tanimura, A., McGregor, D. H., and Anderson, H. C. (1986b) Calcification in atherosclerosis. II. Animal studies. J. Exp. Pathol. 2:275-297.

Van Belle, H. (1976) Alkaline phosphatase. I. Kinetics and inhibition by levamisole of purified isoenzymes from humans. Clin. Chem. 22: 972-976.

Harmey, D., Hessle, L., Narisawa, S., Johnson, K., Terkeltaub, R. and Millán, J. L. Concerted regulation of inorganic pyrophosphate and osteopontin by Akp2, Enpp1 and Ank: An integrated model of the pathogenesis of mineralization disorders. Am. J. Pathol. 164: 1199-1209 (2004).

Kozlenkov, A., Hoylaerts, M. F., Ny, T., Le Du, M-H. And Millán, J. L. Residues determining the binding specificity of uncompetitive inhibitors to tissue-nonspecific alkaline phosphatase. J. Bone Min Res. 19: 1862-1872 (2004).

Narisawa, S., Wennberg, C., and Millán, J. L. Abnormal vitamin B6 metabolism causes multiple abnormalities, but not impaired bone mineralization in alkaline phosphatase knock-out mice. J. Pathol. 193: 125-133 (2001).

Le Du, M-H and Millán, J. L. Structural evidence of functional divergence in human alkaline phosphatases. J. Biol. Chem. 277: 49808-49814 (2002).

Anderson, H. C., Sipe, J. E., Hessle, L., Dhamayamraju, R., Atti, E., Camacho, N. P. and Millán, J. L. Impaired calcification around matrix vesicles of growth plate and bone in alkaline phosphatase-deficient mice. Am. J. Pathol. 164: 841-847 (2004).

Anderson H C., Harmey D., Camacho N P., Garimella R., Sipe J B., Tague S., Bi, X., Johnson K., Terkeltaub R. and Millán J L. Sustained Osteomalacia of Long Bones Despite Major Improvement in Other Hypophosphatasia-Related Mineral Deficits in TNAP/NPP1 Double Deficient Mice. Am. J. Pathol. 166: 1711-1720 (2005).

Fedde, K. N., Blair, L., Silverstein, J., Weinstein, R. S., Waymire, K., MacGregor, G. R., Narisawa, S., Millán, J. L., and Whyte, M. P. Alkaline phosphatase knockout mice recapitulate the metabolic and skeletal defects of infantile hypophosphatasia. J. Bone Min. Res. 14: 2015-2026 (1999)

Johnson, K., Goding, J., Van Etten, D., Sali, A., Hu, S-I, Farley, D., Krug, H., Hessle, L., Millán, J. L., and Terkeltaub, R. Linked deficiencies in extracellular inorganic pyrophosphate (PPi) and osteopontin expression mediate pathologic ossification in PC-1 null mice. J. Bone Min. Res. 18: 994-1004 (2003).

Le Du, M. H., Stigbrand, T., Taus sig, M. J., Menez, A., and Stura, E. A., (2001) Crystal Structure of alkaline phosphatase form human placenta at 1.8 resolution. Implication for substrate specificity, J. Biol. Chem. 276: 9158-9165.

Doellgast G J. Fishman W H. (1977) Inhibition of human placental-type alkaline phosphatase variants by peptides containing L-leucine. Clin. Chim. Acta. 15:449-454.

Fishman, W. H. (1974) Am. J. Med. 56, 617-650.

Muliover, R. A., Plotkin, L. I., and Harris, H. (1978) Ann Hum. Genet. 421-13.

Kozlenkov A, Manes T, Hoylaerts M F, Millán J L, (2002) Function assignment to conserved residues in mammalian alkaline phosphatases. J Biol Chem 277:22992-22999.

We claim:

1. A TNAP modulation target having a polynucleotide sequence substantially similar to the sequence comprising SEQ ID NO: 3 with the proviso that residues 108, 109, 120, 166, 168, 371, 434 and 443 are not substituted and maintain their native conformation around the catalytic Zn1 ion.

2. The TNAP modulation target of claim 1 wherein the TNAP modulation target is part of a system comprising native TNAP polypeptides, recombinant polypeptides, polypeptide fragments, full length polypeptides, chimeric polypeptides, fusion polypeptides and combinations thereof.

3. A method of screening for agents that modulates TNAP activity towards a TNAP substrate comprising the steps of:
a. providing a system further comprising;
i. a TNAP polypeptide capable of degrading PPi to inorganic phosphate;
ii. a substrate capable of being degraded by a TNAP polypeptide;
iii. a reporter system capable of reporting the interaction of the TNAP polypeptide and the substrate;
b. contacting the TNAP polypeptide with an agent;
c. incubating the TNAP polypeptide and the agent for a sufficient amount of time in the presence of the TNAP substrate;
d. measuring the effect said agent has on TNAP activity towards the TNAP substrate;
e. comparing the measured effect from step d to the TNAP activity on the TNAP substrate in the absence of the agent to determine whether the agent is a modulator of TNAP activity.

4. The method of claim 3 wherein the TNAP polypeptide is an isolated and purified full-length recombinant TNAP polypeptide having a sequence substantially similar to SEQ ID NO: 3 or SEQ ID NO: 1.

5. The method of claim 3 wherein the TNAP polypeptide is an isolated and purified recombinant TNAP fragment comprising a catalytic domain for interacting with the TNAP substrate; a modulation target for interaction of the agent with the TNAP polypeptide; and a catalytic Zn1 ion.

6. The method of claim 5 wherein the TNAP fragment comprises residues 108, 109, 120, 166, 168, 371, 434 and 443 properly positioned within a 12 angstrom radius around the Zn1 ion.

7. The method of claim 3 wherein the TNAP substrate comprises p-nitrophenylphosphate, pyridoxal-5′-phosphate or PPi.

8. The method of claim 3 wherein the system provided comprises an in vitro assay or an in vivo assay.

9. The method of claim 8 wherein the system provided is a system comprising cell lines, recombinant cell lines, expression systems, baculovirus systems, animal models, extracted proteins, affinity columns, computer algorithms, in situ docking experiments and multi-well plates.

10. The method of claim 3 wherein the screening method is performed as a high throughput screening method.

11. A modulator discovered by the method of claim 3.

12. A modulator of TNAP activity towards a TNAP substrate wherein the modulator interacts within a TNAP modulation domain of a polypeptide having a sequence that is substantially similar to SEQ ID NO: 1 or SEQ ID NO: 3.

13. The modulator of claim 12 wherein the modulator interacts with a polynucleotide sequence substantially similar to SEQ ID NO: 1 or SEQ ID NO: 3 said interaction being at the amino acids identified affecting modulator binding specificity.

14. The modulator of claim 13 wherein the modulator interacts with amino acids selected from the group consisting of 108, 109, 120, 116, 168, 371, 434 and 443.

15. The modulators of claim 12 wherein said modulators are Compound ID: 5361418, Compound ID: 5804079, Compound ID: 5923412, analogues thereof and derivatives thereof.

16. A method for treating a pathologic condition that is known or suspected of being treatable by modulating TNAP activity by administering an amount of a TNAP modulator sufficient to treat said pathological condition.

17. The method of claim 16 wherein the pathologic condition comprises arterial calcification, arthritis, aneurysm, aging, diabetes, renal failure or aortic stenosis.

18. The method of claim 17 wherein a modulator of TNAP that is known to inhibit TNAP is administered in an amount sufficient to treat arterial calcification, arthritis, aneurysm, aging, diabetes, renal failure or aortic stenosis.

19. The method of claim 16 wherein the modulator comprises a peptide, polypeptide, peptidomimetic, non-peptidyl compound, carbohydrate, lipid, a synthetic compound, a natural product, an antibody or antibody fragment, a small organic molecule, a small inorganic molecule, a nucleotide sequence, and pharmaceutical formulations thereof.

20. The method of claim 16 wherein the modulator is selected from the group consisting of Compound ID: 5361418, Compound ID: 5804079, Compound ID: 5923412, analogues thereof, derivatives thereof and pharmaceutical formulations thereof.


SEQUENCE LISTING (3 sequences)

SEQ ID NO: 1 - protein, 524 aa, Homo sapiens
MISPFLVLAIGTCLTNSLVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSAGSLAAGPLLLALALYPLSVLF

SEQ ID NO: 2 - DNA, 2580 bp, Homo sapiens
tcgggccccgcggccgcctttataaggcggcgggggtggtggcccgggccgcgttgcgctcccgccactccgcgcccgctatcctggctccgtgctcccacgcgcttgtgcctggacggaccctcgccagtgctctgcgcaggattggaacatcagttaacatctgaccactgccagcccaccccctcccacccacgtcgattgcatctctgggctccagggataaagcaggtcttggggtgcaccatgatttcaccattcttagtactggccattggcacctgccttactaactccttagtgccagagaaagagaaagaccccaagtactggcgagaccaagcgcaagagacactgaaatatgccctggagcttcagaagctcaacaccaacgtggctaagaatgtcatcatgttcctgggagatgggatgggtgtctccacagtgacggctgcccgcatcctcaagggtcagctccaccacaaccctggggaggagaccaggctggagatggacaagttccccttcgtggccctctccaagacgtacaacaccaatgcccaggtccctgacagcgccggcaccgccaccgcctacctgtgtggggtgaaggccaatgagggcaccgtgggggtaagcgcagccactgagcgttcccggtgcaacaccacccaggggaacgaggtcacctccatcctgcgctgggccaaggacgctgggaaatctgtgggcattgtgaccaccacgagagtgaaccatgccacccccagcgccgcctacgcccactcggctgaccgggactggtactcagacaacgagatgccccctgaggccttgagccagggctgtaaggacatcgcctaccagctcatgcataacatcagggacattgacgtgatcatggggggtggccggaaatacatgtaccccaagaataaaactgatgtggagtatgagagtgacgagaaagccaggggcacgaggctggacggcctggacctcgttgacacctggaagagcttcaaaccgagatacaagcactcccacttcatctggaaccgcacggaactcctgacccttgacccccacaatgtggactacctattgggtctcttcgagccaggggacatgcagtacgagctgaacaggaacaacgtgacggacccgtcactctccgagatggtggtggtggccatccagatcctgcggaagaaccccaaaggcttcttcttgctggtggaaggaggcagaattgaccacgggcaccatgaaggaaaagccaagcaggccctgcatgaggcggtggagatggaccgggccatcgggcaggcaggcagcttgacctcctcggaagacactctgaccgtggtcactgcggaccattcccacgtcttcacatttggtggatacaccccccgtggcaactctatctttggtctggcccccatgctgagtgacacagacaagaagcccttcactgccatcctgtatggcaatgggcctggctacaaggtggtgggcggtgaacgagagaatgtctccatggtggactatgctcacaacaactaccaggcgcagtctcctgtgcccctgcgccacgagacccacggcggggaggacgtggccgtcttctccaagggccccatggcgcacctgctgcacggcgtccacgagcagaactacgtcccccacgtgatggcgtatgcagcctgcatcggggccaacctcggccactgtgctcctgccagctcggcaggcagccttgctgcaggccccctgctgctcgctctggccctctaccccctgagcgtcctgttctgagggcccagggcccgggcacccacaagcccgtgacagatgccaacttcccacacggcagcccccccctcaaggggcagggaggtgggggcctcctcagcctctgcaactgcaagaaaggggacccaggaaaccaaagtctgccgcccacctcgctcccctctggaatcttccccaagggccaaacccacttctggcctccagcctttgctccctccccgctgccctttggccaccagggtagatttctcttgggcaggcagagagtacagactgcagacattctcaaagcctcttatttttctagcgaacgtatttctccagacccagaggccctgaagcctccgtggaacattgtggatctgaccctcccagtctcatctcctgaccctcccactcccatctccttacctctggaaccccccaggccctacaatgctcatgtccctgtccccaggcgagccctccttcaggggagttgaggtctttctcctcaggacaaggccttgctcactcactcactccaagaccaccagggtcccaggaagccggtgcctgggtggccatcctacccagcgtgcccaggccgggaagagccacctggcagggctcacactcctgggctctgaacacacacgccagctcctctctgaagcgactctcctgtttggaacggcaaaaaaaaatttttttttctctttttggtggtggttaaaagggaacacaaaacatttaaataaaactttccaaatatttccgagg

SEQ ID NO: 3 - protein, 507 aa, Homo sapiens
LVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSAGSLAAGPLLLALALYPLSVLF